(19) United States
(12) Patent Application Publication
(10) Pub. No.: US 2014/0171458 A1
(43) Pub. Date: Jun. 19, 2014

(54) INTRANASAL NALTREXONE

(71) Applicant: 3B Pharmaceuticals, Inc., Parker, CO (US)

(72) Inventors: Darby C. Brown, Parker, CO (US); Jason Montgomery, Highlands Ranch, CO (US); Beau Gertz, Denver, CO (US)

(73) Assignee: 3B Pharmaceuticals, Inc., Parker, CO (US)

(21) Appl. No.: 14/106,257

(22) Filed: Dec. 13, 2013

Related U.S. Application Data
(60) Provisional application No. 61/736,951, filed on Dec. 13, 2012.

(51) Int. Cl. A61K 31/485

(52) U.S. Cl. CPC A61K 31/485
USPC 514/282

(57) ABSTRACT

A stable transmucosal composition consisting essentially of naltrexone hydrochloride in water is provided. The composition is surprisingly stable at room temperature and can be used for simple, rapid and effective opioid overdose rescue.

### CROSS-REFERENCES TO RELATED APPLICATIONS

This application claims the benefit of U.S. Provisional Application No. 61/736,951, filed Dec. 13, 2012, which is incorporated herein by reference.

### FIELD

A stable composition consisting essentially of naltrexone hydrochloride in water is provided. The composition is appropriate for transmucosal administration. The naltrexone hydrochloride composition is surprisingly stable at room temperature and can be used for simple, rapid and effective opioid overdose rescue.

### BACKGROUND

Use of illicit street drugs such as heroin and opium is a widespread problem. Opioid prescription drug abuse and dependence is increasing. Opioids (opioid receptor agonists) are easily available because they are commonly prescribed for postsurgical pain relief, management of acute or chronic pain, relief of cough and relief of diarrhea.

Current medical guidelines for treatment of opioid overdose include administration of naloxone, an opioid receptor antagonist. Emergency medical personnel and community based programs targeting high risk individuals rely on injectable formulations of naloxone.

Unfortunately, Naloxone has been periodically under FDA shortage designation. A recent CDC report stated about 44% of community programs reported problems obtaining naloxone within the “past few months”. The most frequent reported reasons were the cost of naloxone relative to available funding and the inability of suppliers to fill orders. Centers for Disease Control and Prevention, Morbidity and Mortality Weekly Report, Community-based opioid overdose prevention programs providing naloxone-United States, 2010. Feb. 17, 2012; 61(6): 101-105.

Clearly, an alternative, simple, rapid acting dosage form of an opioid receptor antagonist is desirable for administration to a patient with suspected opioid overdose by first responders, emergency room personnel, or for use in community based programs.

### SUMMARY

A stable composition consisting essentially of an opioid receptor antagonist in water is provided. The naltrexone hydrochloride composition is appropriate for transmucosal administration. In some embodiments, a method is provided for treating an opioid receptor agonist overdose comprising administering intranasally the naltrexone hydrochloride composition.

In some embodiments, a kit containing at least one ready to use, single dose intranasal spray consisting essentially of naltrexone hydrochloride in water is provided.

In some embodiments, the disclosure provides a pharmaceutical composition consisting essentially of an opioid receptor antagonist, or a salt thereof, and water, the composition capable of maintaining at least about 90%, or at least about 95%, of said naltrexone hydrochloride in undegraded form after storage for at least 90 days, or at least 180 days, at room temperature. In some embodiments, the opioid receptor antagonist naltrexone hydrochloride is present in the composition in an amount between about 0.001 mg/mL and 20 mg/mL; 0.01 mg/mL and 10 mg/mL; 0.5 mg and 5 mg, 1 mg/mL and 3 mg/mL; or about 2 mg/mL.

In some embodiments, the disclosure provides a composition comprising an opioid receptor antagonist and water wherein the composition does not contain a preservative, buffering agent, tonicity agent, or pH adjusting agent. In some embodiments, the disclosure provides a composition consists essentially of naltrexone hydrochloride dissolved in water at a concentration in the range of from about 0.001 mg/mL to about 20 mg/mL, about 0.01 mg/mL to about 10 mg/mL, about 0.5 mg/mL to about 5 mg/mL, about 1 mg/mL to about 3 mg/mL, or about 2 mg/mL in sterile water or water for injection. In some embodiments, no buffer is employed in the composition.

In some embodiments, the disclosure provides a method for treating a patient in need thereof, the method comprising administering intranasally to the patient a first dose of a pharmaceutical composition consisting essentially of naltrexone hydrochloride and water; wherein the composition is capable of maintaining at least about 90% of said naltrexone hydrochloride in undegraded form after storage for 90 days, or preferably, 180 days, at room temperature. In some embodiments, the composition is administered as an intranasal spray or an intranasal drop.

In some embodiments, the disclosure provides a method for treating a patient in need thereof, the method comprising administering to the patient a first dose of a pharmaceutical composition consisting essentially of naltrexone hydrochloride and water; wherein the composition is capable of maintaining at least about 90% of said naltrexone hydrochloride in undegraded form after storage in a sealed container for at least 90 days, or preferably, at least 180 days, at room temperature. In some embodiments, the composition is administered transmucosally to the patient. In some embodiments, the transmucosal administration is selected from intranasal, buccal, sublingual, vaginal, ocular and rectal route of administration. In a preferred embodiment, the composition is administered intranasally to the patient.

In some embodiments, if the patient does not respond to the first dose, the method further comprises a step of administering a second dose of the composition intranasally to the patient.

In some embodiments, the second dose is administered if no response is detected following administration of the first dose within a range of from about 1 minute to about 10 minutes following administration of the first dose. In some embodiments, the second dose of the composition is the same or greater than amount of naltrexone hydrochloride as, the first dose.

In some embodiments, the patient in need of treatment is suffering from an opioid receptor agonist overdose, a suspected opioid receptor agonist overdose, Crohn's disease, irritable bowel syndrome, fibromyalgia, neuropathic pain, chronic headache, postoperative dental pain, or an opioid side effect selected from constipation, drowsiness, urinary retention, nausea and vomiting, or pruritus. In some embodiments, the patient in need thereof is suffering from, or suspected of suffering from, an opioid agonist overdose.

In some embodiments, the disclosure provides a kit comprising one or more sealed containers, each container filled with a single dose volume of a composition consisting essentially of naltrexone hydrochloride and water, the composition capable of maintaining at least about 90% of said naltrexone hydrochloride in undegraded form after storage for 90 days at room temperature. In some embodiments, the containers in the kit are fitted with, or can be fitted to, a mucosal atomization device (MAD) for intranasal spray administration of the composition to a patient in need thereof.

### BRIEF DESCRIPTION OF THE DRAWINGS

FIG. 1 shows a graph of average blood levels of free Naltrexone (ng/mL) of 8 subjects at various time points (15, 30, 60 and 120 minutes) following intranasal administration of 1 mL of the composition according to Example 1, consisting essentially of naltrexone hydrochloride and water. Average blood levels appear to decline after about 30 minutes post-administration; however, average blood levels of naltrexone remain above about 2 ng/mL from at least about 15 minutes to about 60-120 minutes post-administration.

FIG. 2 shows a graph of average blood levels of free 6-beta-naltrexol (ng/mL), a naltrexone metabolite, of 8 subjects at various time points (15, 30, 60 and 120 minutes) following intranasal administration of 1 mL of the composition according to Example 1, consisting essentially of naltrexone hydrochloride and water. Blood levels appear to increase over time to an average of 2.4 ng/mL at 120 minutes post-administration.

### DETAILED DESCRIPTION

A stable, intranasal composition consisting essentially of naltrexone hydrochloride in water is provided. The intranasal spray formulation is surprisingly stable when stored at room temperature and can be used for simple, rapid and effective opioid overdose rescue.

Opioid Agonists.

Opioids (opioid receptor agonists) are commonly prescribed for postsurgical pain relief, management of acute or chronic pain, relief of cough and relief of diarrhea.

Opioids are included in a number of prescription medications including OXYCONTIN (oxycodone) and VICODIN (hydrocodone). Opioid agonists bind to various opioid receptors in the brain and spinal cord, blocking the perception of pain. In the short term opioids alleviate pain, cause drowsiness, constipation and depressed respiration, depending on dose. Long term use of opioids can result in physical dependence and addiction.

Opioid agonists in prescription medications include, but not limited to, oxycodone (e.g., OXYCONTIN, ROXIDODONE, TYLOX, PERCODAN, PERCOCET), hydrocodone (e.g., VICODIN, LORTAB, LORCET), hydromorphone (e.g., DILAUDID), meperidine (e.g., DEMEROL), propoxyphene (e.g., DARVON, WYGESIC), diphenoxylate (e.g., LOMOTIL), morphine (e.g., KADIAN, AVINZA, MS CONTIN), codeine (e.g., TYLENOL 3, PHENAPHEN), butorphanol (e.g., STADOL), fentanyl (e.g., DURAGESIC, ACTIQ), tramadol (e.g., ULTRAM), pentazocine (e.g., TALACEN, TALWIN), and methadone (e.g., DOLOPHINE).

Naturally occurring opioids include opium and morphine. Morphine is the primary active in opium.

Semisynthetic opioids include heroin, oxycodone, oxymorphone, and hydrocodone.

Synthetic opioids include buprenorphine, methadone, fentanyl, alfentanil, levorphanol, meperidine, and propoxyphene.

Repeated use of prescription or illicit opioids causes not only dependence, but also tolerance where the patient must take an increasing dose to maintain the same effect. Certain prescriptions therefore contain high doses of opioid agonists which can be very dangerous if used inappropriately, particularly if taken with alcohol or other CNS respiratory depressants. Crushing and snorting opioid prescription medications, or mixing with other drugs can cause overdose, which can be fatal. Illicit street drug opioids such as heroin are also commonly associated with overdose situations. Opioid overdose can cause severe respiratory depression and death.

Opioid Overdose.

Signs of opioid overdose in a patient include any of decreased level of consciousness, loss of consciousness, pinpoint pupils, reduced heart rate, reduced breathing rate, seizures, muscle spasms, and blue nails and lips caused by insufficient oxygen in the blood. A patient presenting with coma with unknown etiology with respiratory depression and/or constricted pupils is suspected of suffering from an opioid narcotic or synthetic narcotic overdose.

Current medical guidelines for treatment of opioid overdose include assessing patient to clear airway; providing support ventilation, if needed; assessing and supporting cardiac function; providing IV fluids; monitoring vital signs and cardiopulmonary status; and IV administration of naloxone (e.g., NARCAN®, Endo Pharmaceuticals), an opioid receptor antagonist. Naloxone dosing for reversing opioid overdose is 0.4-2.0 mg/dose by intravenous (IV) or intramuscular (IM) injection. Typical emergency services protocols give medics the authority to treat with 2 mg naloxone intravenously/intramuscularly, or 4 mg via endotracheal tube, and to repeat the dose if no response is observed. Vilke et al., 2003, Assessments for deaths in out-of-hospital heroin overdose patients treated with naloxone who refuse transport, Acad Emerg Med, August 2003, 10(8): 893-896.

Intravenous Naloxone works within about two to eight minutes to restore breathing, bringing the victim to consciousness. Naloxone works to block brain cell receptors activated by opioid receptor agonists such as oxycodone, hydrocodone and heroin. Naloxone works to reverse the effects of opioids on the brain and respiratory system in order to prevent the ultimate adverse event, death. Naloxone is known to be poorly absorbed sublingually or orally, but well absorbed intravenously.

Community-based distribution programs have been instituted in several states in order to provide overdose training and take-home doses of naloxone, to be administered nasally or by intramuscular injection, to those deemed high-risk for overdose. Several states have enacted legislation to allow legal prescription to a person at-risk.

Unfortunately, Naloxone has been periodically under FDA shortage designation. A recent CDC report stated about 44% of community programs reported problems obtaining naloxone within the “past few months”. The most frequent reported reasons were the cost of naloxone relative to available funding and the inability of suppliers to fill orders. Centers for Disease Control and Prevention, MMWR 2012; 61(6): 101-105.

In some embodiments, a method for treating or preventing an opioid receptor agonist overdose is provided comprising transmucosally administering a composition consisting essentially of naltrexone hydrochloride and water; wherein the composition is capable of maintaining at least about 90% of said naltrexone hydrochloride in undegraded form after storage in a sealed container for at least 90 days, or preferably, at least 180 days, at room temperature.

In some embodiments, a method for treating an opioid receptor agonist overdose is provided comprising intranasally administering a composition consisting essentially of naltrexone hydrochloride and water; wherein the composition is capable of maintaining at least about 90% of said naltrexone hydrochloride in undegraded form after storage in a sealed container for at least 180 days, at room temperature.

Opioid Antagonists.

In some embodiments, the compositions and methods of the disclosure employ a composition comprising one or more centrally-acting opioid receptor antagonists. Opioid receptor antagonists include naltrexone, naloxone, nalmefene, naloxonazine, nor-binaltorphimine, and naltrindole. Opioid receptor antagonists displace opioid agonists from their neuroreceptors and block opioids from binding and activating those receptors. Short acting antagonists like naloxone are used to quickly reverse toxic effects of opioid overmedication or overdose. In some embodiments, a composition consisting essentially of an opioid receptor antagonist and water is provided for intranasal administration to treat a patient suffering from opioid receptor agonist overdose or suspected of suffering from opioid receptor agonist overdose. In some embodiments, the opioid antagonist is in the form of a pharmaceutically acceptable salt. In some embodiments, the opioid antagonist is naloxone or naltrexone. In some embodiments, the opioid antagonist is naltrexone hydrochloride.

Naloxone and Naltrexone are opioid receptor antagonists used to treat acute opioid overdose and help control alcohol/opioid long term dependence. In some embodiments, the intranasal opioid receptor antagonist compositions disclosed herein are used to treat opioid overdose in a patient in need thereof.

In some embodiments, the disclosure provides a method for treating opioid overdose, or suspected opioid overdose, comprising administering intranasally a composition consisting essentially of naltrexone hydrochloride and water. In some embodiments, the compositions of the invention are useful for treating overdose or an adverse effect of an opioid. In some embodiments, the disclosure provides a method for treating overdose or an adverse effect of an opioid, the method comprising administering a composition consisting essentially of naltrexone hydrochloride and water, wherein the opioid is selected from the group consisting of heroin, oxycodone, oxymorphone, hydrocodone, hydromorphone, opium, codeine, morphine, butorphanol, buprenorphine, meperidine, methadone, fentanyl, alfentanil, levorphanol, meperidine, pentazocine, propoxyphene, diphenoxylate, tramadol.

Other Naltrexone and Naloxone Dosage Forms.

Oral dosage forms of opioid antagonists are available for regular administration and control of opioid and alcohol addiction. However, in the case of an opioid overdose, delivery of opioid antagonists in an oral dosage form is not desirable for a number of reasons. The patient may not be able to ingest an oral dosage form, or—even if ingested—the oral dosage form may not be adequately delivered, because the patient has lost consciousness, because peristalsis is slowed, or because patient may suffer from vomiting. Oral dosage forms also suffer from delayed bioavailability. Other opioid antagonist dosage forms are available.

SUBOXONE® sublingual film (buprenorphine and naloxone, sublingual film; Reckitt Benckiser; 4:1 ratio) is available in various dosage strengths. Suboxone sublingual film is indicated only for maintenance treatment of opioid dependence. Suboxone is also available in a pill form for treatment of opioid dependence. Buprenorphine is a partial agonist at the opioid mu receptor and an antagonist at the kappa opioid receptor. Naloxone has limited bioavailability by sublingual or oral routes, but was added to buprenorphine to deter intravenous abuse of this drug. Use of SUBOXONE in acute opioid overdose is not recommended.

VIVITROL® (Naltrexone hydrochloride, intramuscular, 380 mg/vial, Alkermes) is available as a suspension for extended release intramuscular injection. VIVITROL is indicated for treatment of alcohol dependence and for prevention of relapse to opioid dependence following opioid detoxification. Extended release intramuscular injection is thought to be beneficial to patient compliance compared to regular oral dosage forms. VIVITROL is administered by intramuscular injection once every four weeks. VIVITROL must not be given intravenously or subcutaneously. VIVITROL is contraindicated in patients receiving or dependent on opioids. Due to the high relative dose and extended release formulation, VIVITROL is not useful for opioid overdose treatment.

Naloxone injection (NARCAN®, Endo Pharmaceuticals) comes as a sterile solution to inject intravenously, intramuscularly, or subcutaneously. Three concentrations are available at 0.02 mg, 0.4 mg, and 1 mg per mL. The 0.02 mg/mL concentration is unpreserved with 9 mg/mL sodium chloride, and pH is adjusted to 3.5 with hydrochloric acid. The 0.4 mg/mL vial contains 8.6 mg/mL sodium chloride, and 2 mg/mL methylparaben and propylparaben as preservatives in a ratio of 9:1. The 1.0 mg/mL vial contains 8.35 mg/mL of sodium chloride, 2 mg/mL methylparaben and propylparaben as preservatives in a ratio of 9:1. NARCAN is indicated for the complete or partial reversal of opioid depression, including respiratory depression, induced by natural and synthetic opioids, including propoxyphene, methadone and certain mixed agonist/antagonist analgesics: nalbuphine, pentazocine, butorphanol, and cyclazocine. NARCAN is also indicated for diagnosis of suspected or known acute opioid overdosage. When administered intravenously, the onset of action is generally less than 2 minutes. The onset is slightly less rapid when administered intramuscularly (IM) or subcutaneously (SQ).

Naloxone injection can cause side effects such as pain, burning or redness at injection site, nausea, vomiting, uncontrollable shaking, pain burning or numbness in hands or feet, sweating and flushing. Some side effects can be serious including rapid, pounding or irregular heartbeat, chest pain, shortness of breath, difficulty breathing or wheezing, hallucination, loss of consciousness and seizures. Naloxone injection can also cause withdrawal symptoms such as body aches, diarrhea, fast heartbeat, fever, runny nose, sweating, nausea, nervousness, restlessness, irritability, and stomach cramps.

Naltrexone has better oral bioavailability than naloxone. Although naloxone is well absorbed, it is subject to extensive first pass metabolism. Both naltrexone and naloxone undergo hepatic metabolism. Naltrexone has a longer duration of action than naloxone. Both naltrexone and naloxone can act at all classes of opioid receptor, particularly the opioid μ receptor. Typical naltrexone doses used for addiction therapy (50-100 mg/day orally) and naloxone for reversing opioid overdose (0.4-1.0 mg/dose IV or IM) are some cases different from the dosing used in pain management applications.

In general, single dose range of opioid receptor antagonists, e.g., naltrexone, in the 1 mg to 5 mg range are referred to as “low dose”, where doses less than 1 mg are referred to as “ultralow dose.” Doses in the 25 to 150 mg per day dose range are said to be “high dose”. High dose naltrexone is typically employed in the treatment of alcohol dependence. Yoon et al., 2011 Safety, tolerability, and feasibility of high-dose naltrexone in alcohol dependence: an open-label study. Hum Psychopharmacol 2011 March, 26(2): 125-132.

Naltrexone is an opioid antagonist derived by the substitution of the N-methyl group of oxymorphone with a cyclopropyl group. The structure of naltrexone is similar to naloxone, but is has a higher oral efficiency and a longer duration of action. A single oral dose reaches peak plasma concentration in 1-2 hours with an apparent half-life of about 14 hours. Naltrexone is a pure antagonist at the opioid mu receptor with no intrinsic agonist effects and can effectively block the effects of substantial opioid analgesics. It has been reported that using 25 mg intravenous heroin challenges in former drug addicts, a 100 mg dose of naltrexone provided 96% blockade at 24 hours, 86.5% at 48 hours and 46.6% at 72 hours. Discontinuation produces few signs and symptoms. However, long term use increases the concentration of opioid receptors in the brain and can produce a temporary exaggeration of responses to the subsequent administration of opioid agonists. Vickers et al., BMJ 2006, January 21, Naltrexone and problems in pain management. 332(7534): 132-133.

Naltrexone is used to help patients avoid relapse after they have been detoxified from opioid dependence. In this usage, the patient must be detoxified prior to treating to avoid severe withdrawal symptoms. Naltrexone is given orally at 50 mg per day or up to 200 mg twice a week. Use of naltrexone at these dosages requires testing the patients liver enzymes prior to chronic treatment. Naltrexone can cause hepatocellular injury when given in excessive doses and is contraindicated in acute hepatitis or liver failure, so limited exposure is desirable.

Naltrexone has a molecular weight of 341.4 g/mol and is freely soluble in water when in the form of a salt. Naltrexone hydrochloride is soluble in water to the extent of about 100 mg/mL. Naltrexone is a pure opioid antagonist. By oral administration, naltrexone is subject to significant first pass metabolism with oral bioavailability estimated at up to 40%. Naltrexone is most commonly available as a hydrochloride salt, but any pharmaceutically acceptable naltrexone salt can be employed in the compositions.

Other Uses of Opioid Antagonists.

Paradoxically, it has been reported that opioid receptor antagonists can act as adjuvants for enhancing rather than attenuating analgesic effects of opioids like morphine and oxycodone, and others. Opioid receptor antagonists can also be employed in low dose compositions as monotherapy and have been employed for better managing certain chronic pain conditions. Leavitt 2009, Opioid Antagonists, Naloxone and Naltrexone-Aids for Pain Management, Pain Treatment Topics, <Pain-Topics.org> March 2009: 1-16. Naloxone has been shown to have an analgesic effect at low doses (e.g., 2 mg), but not at higher doses (e.g., 7.5 mg and 10 mg). Sloan and Hamann 2006, J Opioid Manage 2006; 2 (5):295-304. The precise mechanism is not known, but it has been proposed that transient blockade of opioid receptors by low doses of antagonists stimulates upregulation of mu opioid receptors in areas of the brain responsible for pain responses and that the body responds to temporary opioid receptor blockade by producing increased amounts of endorphins, which are endogenous opioid agonists. Leavitt 2009.

In some embodiments, the transmucosal opioid antagonist compositions disclosed herein are used for chronic pain management in a patient in need thereof. In some embodiments, the intranasal naltrexone hydrochloride compositions disclosed herein are used for chronic pain management in a patient in need thereof.

In some embodiments, the intranasal opioid antagonist compositions disclosed herein are used for chronic pain management in a patient in need thereof. In some embodiments, the intranasal naltrexone hydrochloride compositions disclosed herein are used for chronic pain management in a patient in need thereof.

Naloxone and naltrexone are sometimes used at low dose or ultra-low dose in combination with opioid agonists to reduce opioid tolerance and side effects. Chronic morphine use has been shown to cause the mu receptor to switch its coupling from Gi/o to Gs, provoking excitatory signaling, but ultra-low doses of naloxone or naltrexone prevent this switch, which is thought to attenuate the addictive properties of morphine and allow for lower effective doses. Arbuck et al., Management of opioid tolerability and related adverse effects, J. Medicine May/June 2010 3(1) 1-10. In some embodiments, the composition consisting essentially of an opioid receptor antagonist and water is useful for co-administration with an opioid agonist to a patient in need thereof, for example, as a treatment for chronic pain.

It has also been reported that relatively low doses of opioid receptor antagonists are useful for attenuating adverse effects, or side effects, of administration of opioids, for example, constipation, drowsiness, nausea and vomiting, headache and pruritis. Headaches are a common adverse effect with opioids. Opioids are known to induce headaches in chronic headache patients. Opioid agonists are known to elevate cerebralspinal fluid pressure (CSF pressure elevation) and cause retention of CO₂ due to respiratory depression. Arbuck et al., 2010, J. Medicine 3(1) 1-10.

In some embodiments, compositions and methods are provided for attenuating adverse effects, or side effects, of opioid administration. In some embodiments, the transmucosal composition comprising naltrexone hydrochloride is co-administered with oral opioids for attenuation of undesirable side effects. In some embodiments, a method for treating or preventing opioid receptor agonist side effects is provided comprising transmucosally administering the composition to a patient in need thereof for attenuation of one or more undesirable side effects including, for example, constipation, drowsiness, nausea and vomiting, headache and pruritis. In some embodiments, the composition is administered by intranasal or sublingual administration.

In some embodiments, a method for treating or preventing opioid receptor agonist side effects is provided comprising administering intranasally the composition to a patient in need thereof for attenuation of one or more undesirable side effects including, for example, constipation, drowsiness, nausea and vomiting, headache and pruritis.

In some embodiments, a method for treating or preventing opioid receptor agonist side effects is provided comprising administering sublingually the composition to a patient in need thereof for attenuation of one or more undesirable side effects including, for example, constipation, drowsiness, nausea and vomiting, headache and pruritis.

Low dose naltrexone (LDN) has been reported anecdotally to reduce symptoms of chronic headache. In some embodiments, the intranasal opioid antagonist compositions disclosed herein are used for treatment of chronic headache in a patient in need thereof. In some embodiments, the intranasal naltrexone hydrochloride compositions disclosed herein are used for treatment of chronic headache in a patient in need thereof.

In some embodiments, the disclosure provides a composition consisting essentially of naltrexone hydrochloride and water that is useful for treating a patient suffering from Crohn's disease. Naltrexone has been tested as a monotherapy to treat Crohn's disease, irritable bowel syndrome (IBS), fibromyalgia, and neuropathic pain. One clinical trial in 2007 treated patients suffering from Crohn's disease with 4.5 mg/day naltrexone for 12 weeks in an open-label study. Statistically significant improvements were found in CDAI (Crohn's Disease Activity Index) scores, quality of life indicators, increased rates of remission and decreases in inflammatory markers in serum. See, for example, Leavitt, Opioid Antagonists. Naloxone & Naltrexone-Aids for Pain Management. March 2009. Pain Treatment Topics, Pain-Topics.org. 1-16. In some embodiments, the composition consisting essentially of an opioid receptor antagonist and water is useful for treating a patient suffering from Crohn's disease.

In some embodiments, the composition consisting essentially of an opioid receptor antagonist and water is useful for treating a patient suffering from irritable bowel syndrome. Irritable bowel syndrome is a symptom based diagnosis characterized by chronic abdominal pain, discomfort, bloating and alteration of bowel habits. In one open-label study, patients suffering from IBS received 0.5 mg/day oral naltrexone daily for 4 weeks. Primary outcomes were number of pain free days, and overall symptom relief including degree of abdominal pain, stool urgency, consistency, and frequency. Global assessment improved in 76% of 42 patients. Kariv et al., Low-dose naltrexone for the treatment of irritable bowel syndrome: a pilot study. Dig Dis Sci 2006 December; 51(12):2128-2133. In some embodiments, the composition consisting essentially of naltrexone hydrochloride and water is useful for treating a patient suffering from irritable bowel syndrome.

In some embodiments, the composition consisting essentially of naltrexone hydrochloride and water is useful for treating a patient suffering from fibromyalgia. Fibromyalgia is a disorder characterized primarily by chronic widespread pain. Other symptoms include fatigue, sleep irregularities, bowel abnormalities, anxiety and mood dysfunction. There is good evidence to suggest that naltrexone has a neuroprotective role and may be potentially effective treatment for diseases like fibromyalgia. Deshpande et al., 2011, A control engineering approach for designing an optimized treatment for fibromyalgia, Proc Am Control Conf 2011, Jun. 29; 2011:4798-4803. One single-blind, crossover clinical trial treated patients suffering from fibromyalgia with a 4.5 mg oral daily dose of naltrexone. Low dose naltrexone reduced fibromyalgia symptoms in the entire cohort, with a greater than 30% reduction of symptoms over placebo. Younger et al., 2009, Fibromyalgia symptoms are reduced by low-dose naltrexone: a pilot study. Pain Med. 2009; 10(4):663-672. In some embodiments, the composition consisting essentially of an opioid receptor antagonist and water is useful for treating a patient suffering from fibromyalgia

In some embodiments, the composition consisting essentially of an opioid receptor antagonist and water is useful for treating a patient suffering from neuropathic pain. Neuropathic pain may result from disorders of the peripheral nervous system or the central nervous system. Neuropathic pain is characterized by abnormal sensations called dysesthesia (abnormal sense of touch), and pain produced by normally non-painful stimuli. Neuropathic pain qualities include burning or coldness, pins and needles sensations, numbness and itching. Cruciani 2003 described a patient with chronic painful diabetic neuropathy being unsuccessfully treated with methadone. Use of ultralow dose of oral naltrexone 0.002 mg/day improved pain relief and allowed a reduction in methadone dose. Cruciani et al 2003, Ultra-low dose oral naltrexone decreases side effects and potentiates the effects of methadone. J Pain Symptom Manage, June, 25(6):491-4.

In some embodiments, the composition consisting essentially of naltrexone hydrochloride and water is useful for treating a patient suffering from postoperative dental pain. Oral naltrexone alone, either 0.4 mg or 1.0 mg, produced significant analgesic effects compared with placebo for postoperative dental pain in a trial of 90 patients. In some embodiments, the composition consisting essentially of an opioid receptor antagonist and water is useful for treating a patient suffering from postoperative dental pain.

### DEFINITIONS

As used herein, the terms “about” or “approximately” broaden the numerical value. For example, in some cases, “about” or “approximately” refers to +/−10%, of the relevant unit value. Also, the disclosure of ranges is intended as a continuous range including every value between the minimum and maximum values recited.

As used herein, the term “treatment” embraces all the different forms or modes of treatment as known to those of the pertinent art and in particular includes preventive, curative, delay of progression and palliative treatment.

Unless otherwise specified, values expressed as % refer to % w/v.

As used herein, the term “opioid receptor antagonist” includes any substance that selectively blocks an opioid receptor of any type (e.g., mu, delta, kappa, etc.) or subtype (e.g., mu1/mu2). Suitable opioid receptor antagonists for use in the present invention include, but are not limited to, any centrally acting opioid receptor antagonist. In some embodiments, the antagonist is selected from naltrexone, nalmefene, naloxone, naloxonazine, nor-binaltorphimine, naltrindole or combinations thereof. In some embodiments, the opioid receptor antagonist is naltrexone.

The term “subject”, or “patient”, refers to an animal, for example a mammal, such as a human, who is the object of treatment. The patient may also be a domestic production animal, exotic zoo animal, wild animal, or companion animal. The subject, or patient, may be either male or female.

Opioid receptor agonists (sometimes abbreviated as opioid agonists, or opioids) and opioid receptor antagonists (sometimes abbreviated as opioid antagonists) can also be called opiates.

The opioid receptor antagonist may be in free form or in pharmaceutically acceptable salt or complex form. “Pharmaceutically acceptable salts,” or “salts,” include salts of mineral acids such as hydrochloric, hydriodic, hydrobromic, phosphoric, metaphosphoric, nitric and sulfuric acids, as well as salts of organic acids such as formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, betahydroxybutyric, galactaric and galacturonic acids. In some specific embodiments, the hydrochloride salt of naltrexone is employed.

Route of Administration.

The composition is administered transmucosally to the patient. In some embodiments, the transmucosal administration is selected from intranasal, buccal, sublingual, vaginal, ocular and rectal route of administration. In one specific embodiment, the composition is administered sublingually to the patient. In a preferred embodiment, the composition is administered intranasally to the patient.

Intranasal Route of Administration.

Intranasal administration is generally easier for the layperson and has fewer and less severe complications, requires less training and experience than IV drug administration. Intramuscular injections are easy to administer, but drugs administered intranasally can be more rapidly absorbed due to the high volume of potential surface area for absorption, and the drug may be delivered directly to the brain in some species. Intranasal absorption is almost as fast as intravenous administration. Intranasal administration also avoids first pass metabolism of drugs known to be metabolized by the liver, and may be associated with fewer side effects. Other advantages of intranasal administration include avoidance of needle stick injuries, and painless administration.

There are two predominant mechanisms of nasal drug absorption. The first mechanism involves an aqueous route of transport, which is known as the paracellular route. This route is passive and there is generally an inverse correlation between intranasal absorption and the molecular weight of water soluble compounds. Poor bioavailability is sometimes observed for drug with a molecular weight greater than 1,000 g/mol. The second mechanism involves transport through a lipoidal route, also known as a transcellular process. This route of transport is dependent on the lipophilicity of the drug. Drugs can also cross by active transport via a carrier-mediated means or transport through the opening of tight junctions. For example, chitosan, a natural biopolymer from shellfish, is used as a penetration enhancer to open tight junctions between epithelial cells to facilitate transport. In embodiments, the disclosure provides aqueous compositions consisting essentially of an opioid receptor antagonist and water. In some embodiments, the composition does not contain a lipid or a penetration enhancer.

The intranasal compositions disclosed herein can be administered as a spray, drop or intranasal inhalant. The commercial package containing the intranasal formulation can be in a syringe, single dose form, or any drop, spray or nasal inhaler container known in the art.

In some embodiments, the intranasal composition is packaged in a container as a single dose. In some embodiments, the intranasal composition is packaged in a single dose, two dose or three dose container.

In some embodiments, the intranasal composition comprising an opioid receptor antagonist is packaged in a two or three dose container, if the patient does not respond adequately to the first dose by reversal of any one or more of overdose symptoms within a period of time selected from about 1, 2, 3, 4, 5, 6, 7, 8, 9, 10, 15, 20, 25, or 30 minutes following administration of the first dose, a second dose can be administered intranasally. If the patient responds adequately to the first dose, or second dose, for example by achieving altered mental status, the remaining doses can be expelled in an appropriate waste stream and the container is disposed.

In some embodiments, the intranasal composition is administered to a patient in need thereof to reverse the effects of opiate drugs, narcotics, synthetic narcotics, morphine, Dilaudid, Fentanyl, Demeral, Paragoric, Methadone, heroin, Percodan, Tylox, Nubain, Stadol, Talwin, Darvon or any opioid receptor agonist. In some embodiments, one dose of the intranasal composition for administering to a patient in need thereof is 0.1-3 mL of a composition consisting essentially of naltrexone hydrochloride dissolved in water at a concentration in the range of from about 0.001 mg/mL to about 20 mg/mL, about 0.05 mg/mL to about 10 mg/mL, about 0.5 mg/mL to about 5 mg/mL, about 1 mg/mL to about 3 mg/mL, or about 2 mg/mL in sterile water or water for injection. In some embodiments, no buffer is employed in the composition.

In some embodiments, a dose for intranasal administration to a patient in need thereof is selected from 0.5 mL, 1.0 mL, 1.5 mL, 2.0 mL or 2.5 mL of a naltrexone hydrochloride composition for intranasal administration, wherein the composition consists essentially of 0.5, 1.0, 1.5, 2.0, 2.5, 3.0, 3.5, 4.0, 4.5, or 5.0 mg/mL naltrexone hydrochloride and water.

In some specific embodiments, one dose is one mL of intranasal composition, wherein the composition consists essentially of 2 mg/mL naltrexone hydrochloride and water.

In some embodiments, administering naltrexone hydrochloride intranasally one mL of 2.0 mg/mL.

Compositions.

In the art, most preparations intended for intranasal use are in solution form and are administered as nose drops or sprays. Aqueous nasal solutions are typically rendered isotonic to nasal fluids (approximately 0.9% sodium chloride), buffered to maintain drug stability while approximating the normal pH range of the nasal fluids (about pH 5.5-6.5), and stabilized and preserved as required. The antimicrobial preservatives are typically the same as used in ophthalmic solutions. Ansel's Pharmaceutical Dosage Forms and Drug Delivery Systems, 9^{th} Ed., Lippincott, Williams & Wilkins, 2011, p. 547. Nasal irritation is minimized when the pH of the composition is between about 4.5 and 6.5.

Intranasal pharmaceutical compositions comprising an opioid receptor antagonist and water are provided herein. In some embodiments, the opioid receptor antagonist, in the form of a pharmaceutically acceptable salt, is dissolved in water, without a buffer or preservative. In some embodiments, the water is sterile water, or water for injection. For example, naltrexone hydrochloride is dissolved in water at a concentration in the range of from about 0.1 mg/mL to about 20 mg/mL, about 0.5 mg/mL to about 5 mg/mL, about 1 mg/mL to about 3 mg/mL or about 2 mg/mL in sterile water, without preservatives, buffers, tonicity agents, pH adjusters, or surfactants.

It has been surprisingly found that a composition consisting of naltrexone hydrochloride in sterile water is stable when stored at room temperature over a period of at least 90 days, or at least 180 days, even when exposed to ambient humidity and light. In some embodiments, the composition does not contain a preservative. In some embodiments, the composition does not contain a buffer. In some embodiments, the composition does not contain a tonicity agent. In some embodiments, the composition does not comprise a surfactant. In some embodiments, the composition does not comprise a penetration enhancer. In some embodiments, the intranasal composition does not contain a stabilizer. In some embodiments, the intranasal composition does not contain a chelating agent. Surprisingly, a solution of naltrexone hydrochloride in water without a buffer, isotonicity agent, preservative or stabilizer was found to be stable at room temperature for a period of at least 90 days, or at least 180 days.

In some embodiments, the intranasal composition comprising an opioid receptor antagonist and water contains an antioxidant, surfactant, adhesive, pH adjusting agent, stabilizer, osmolarity adjusting agent, preservative, thickening agent, buffering agent, penetration enhancer, chelating agent, sweetening agent, flavoring agent, taste masking agent, or colorant. In some embodiments, the intranasal composition comprising an opioid receptor antagonist and water does not contain an antioxidant, surfactant, adhesive, pH adjusting agent, stabilizer, osmolarity adjusting agent, tonicity agent, preservative, thickening agent, buffering agent, chelating agent, or penetration enhancer.

In some embodiments, the intranasal composition contains an antioxidant. In some embodiments, the intranasal composition contains an antioxidant agent selected from butylated hydroxytoluene, butylated hydroxyanisole, or potassium metabisulfite. In some embodiments, the naltrexone hydrochloride intranasal composition does not contain an antioxidant.

In some embodiments, the intranasal composition of the disclosure comprises an osmolarity adjusting agent or a tonicity agent. Agents that increase tonicity are for example: sodium chloride, dextrose, lactose or mixtures thereof; preferably in amounts between 0.1-5.0% (w/v), preferably 0.1-2.0% (w/v), more preferably 0.1-0.9% (w/v). In some embodiments, the naltrexone hydrochloride intranasal composition disclosed herein does not include an osmolarity adjusting agent or a tonicity agent.

In some embodiments, the composition comprises a stabilizer selected from the group consisting of organic acids, carboxylic acids, acid salts of amino acids, sodium metabisulphite, ascorbic acid and its derivatives, malic acid, isoascorbic acid, citric acid, tartaric acid, sodium sulphite, sodium bisulphate, tocopherol, water- and fat-soluble derivatives of tocopherol, sulphites, bisulphites and hydrogen sulphites, butylated hydroxyanisol (BHA), 2,6-di-t-butyl-alpha-dimethylamino-p-cresol, t-butylhydroquinone, di-t-amylhydroquinone, di-t-butylhydroquinone, butylhydroxytoluene, butylhydroxyanisole, pyrocatechol, pyrogallol, propyl/gallate, and nordihydroguaiaretic acid, phosphoric acids, sorbic and benzoic acids, esters, derivatives and isomeric compounds, and ascorbyl palmitate. In some embodiments, the naltrexone hydrochloride intranasal composition disclosed herein does not contain a stabilizer.

In some embodiments, the intranasal composition contains a penetration enhancer. Penetration enhancers known in the art include cyclodextrins such as carboxymethyl-β-cyclodextrin, hydroxypropyl-β-cyclodextrin, phospholipids, ammonium glycirrhizinoate, tetradecylmaltoside, alkylmaltosides, alkanoylsucroses, chitosan, EDTA, TWEEN 80, SLS, bile salts, ethanol, 10% oleic acid with ethanol, propylene glycol, 10% oleic acid in combination with propylene glycol. Typically, the intranasal penetration enhancer is employed in an amount of from about 0.1% to about 5% w/v in the composition. In some embodiments, the naltrexone hydrochloride intranasal composition does not contain a penetration enhancer.

In some embodiments, the intranasal composition contains a preservative. Certain preservatives are known to cause irritation to the nasal mucosa. In some embodiments, the intranasal compositions do not contain the preservatives benzalkonium chloride, methylparaben, ethylparaben, propylparaben, butylparaben, benzyl alcohol, phenylethyl alcohol, or benzethonium. In some embodiments, the intranasal composition does not contain a preservative.

In some embodiments, the intranasal composition contains a buffering agent. Some buffering agents are known to cause irritation to the nasal mucosa. In some embodiments, the intranasal composition contains buffering agents such as alkali (sodium and potassium) or alkaline earth (calcium and magnesium) salts of carbonate, phosphate, bicarbonate, citrate, borate, acetate, phthalate, tartrate, or succinate. In some embodiments, the intranasal composition does not contain a buffering agent.

In some embodiments, the intranasal composition contains a pH adjusting agent. The pH adjusting agents include one or more of hydrochloric acid and sodium hydroxide. In some embodiments, the naltrexone hydrochloride intranasal composition does not contain a pH adjusting agent.

In some embodiments, the intranasal composition contains an agent that increases viscosity. In some embodiments, the intranasal composition contains a viscosity increasing agent selected from a methylcellulose, carboxymethylcellulose sodium, ethylcellulose, carrageenan, or a carbopol, for example, hydroxypropyl methylcellulose (hypromellose), hydroxyethyl cellulose, hydroxypropyl cellulose, methylcellulose, microcrystalline cellulose, carboxymethylcellulose sodium, xanthan gum or mixtures thereof; preferably in amounts between: 0.01-2.0% (w/v), preferably 0.02-1.0% (w/v), more preferably 0.05-0.5% (w/v). In some embodiments, the intranasal composition does not contain an agent that increases viscosity.

In some embodiments, the intranasal composition comprising an opioid receptor antagonist and water contains a chelating agent wherein the chelating agent is selected from the group consisting of EDTA (ethylene diamine tetraacetic acid), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), 2-(2-amino-2-oxoethyl)aminoethane sulfonic acid (BES), diethylenetriaminepentaacetic acid, bis(aminoethyl)glycolether-N,N,N,N-tetraacetic acid, N-2-acetamido-2-iminodiacetic acid (ADA), N-hydroxyethyliminodiacetic acid (HIMDA), N,N-bis-hydroxyethylglycine (bicine); N-(trishydroxymethylmethyl)glycine (tricine), glycylglycine, iminodiacetic acid, citric acid, tartaric acid, fumaric acid, glutamic acid, aspartic acid mixtures thereof, and salts thereof. In some embodiments, the intranasal composition comprising an opioid receptor antagonist and water does not contain a chelating agent.

In some compositions, the compositions are packaged and stored in single dose containers containing about 0.1, 0.5, 0.7, 1, 1.5, 2.0, 2.5, 3.0, 3.5, or 4.0 mL of the intranasal composition for intranasal administration. In some embodiments, the single dose, two dose or three dose container is a syringe. In some embodiments, the syringe can be fitted with a spray attachment for administration of the formulation as an intranasal spray. In some embodiments, the disclosure provides a method of treating a patient with suspected or known opioid overdose.

In some embodiments, the intranasal pharmaceutical composition comprises an opioid receptor antagonist and water in a composition of the invention, wherein the opioid receptor antagonist is in the form of a pharmaceutically acceptable salt in an amount of about 0.001 mg/mL to about 20 mg/mL, about 0.05 mg/mL to about 10 mg/mL, about 0.5 mg/mL to about 5 mg/mL, about 1 mg/mL to about 3 mg/mL, or about 2 mg/mL. In some embodiments, about 0.1 mL to about 3 mL, about 0.5 to about 2 mL, or about 1 mL of the composition is administered as an intranasal spray to a patient in need thereof. In some embodiments, the opioid receptor antagonist is naltrexone hydrochloride. In some embodiments, the water in the composition is sterile water, or water for injection.

In some embodiments, the intranasal pharmaceutical composition consists essentially of naltrexone hydrochloride and water in a composition of the invention in an amount of about 0.1 mg/mL to about 10 mg/mL, about 0.5 mg/mL to 5 mg/mL, about 1 mg/mL to about 3 mg/mL, or about 2 mg/mL. In some embodiments, about 0.1 mL to about 3 mL, about 0.5 to about 2 mL, or about 1 mL of the composition is administered as an intranasal spray to a patient in need thereof.

In some embodiments, the intranasal pharmaceutical composition consists of naltrexone hydrochloride and water in a composition of the invention in an amount of about 0.1 mg/mL to about 10 mg/mL, about 0.5 mg/mL to 5 mg/mL, about 1 mg/mL to about 3 mg/mL, or about 2 mg/mL. In some embodiments, about 0.1 mL to about 3 mL, about 0.5 to about 2 mL, or about 1 mL of the composition is administered as an intranasal spray to a patient in need thereof.

In some embodiments, the composition comprising naltrexone hydrochloride in water is provided in a single dose container, wherein the delivered dose of naltrexone hydrochloride is selected from about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg of naltrexone hydrochloride in water.

In some embodiments, the intranasal pharmaceutical composition consisting essentially of naltrexone hydrochloride and water in the composition of the invention is administered to a patient in need thereof in a dose selected from an ultra-low dose, low dose, or higher dose range.

In some embodiments, an intranasal spray composition for administration to a patient suffering from opioid overdose, or suspected opioid overdose, contains naltrexone hydrochloride at a pharmaceutically effective dose of 2 mg/mL in sterile water, wherein 1 mL is administered to the patient as an intranasal spray. However, the amount of opioid receptor antagonist administered per dose or the total volume of composition administered will depend on such factors as the nature and severity of the overdose symptoms, the age, weight, and general health of the patient. The dose can be repeated if the patient does not adequately respond by alleviation of one or more symptoms of overdose.

In some embodiments, the intranasal composition consisting of naltrexone hydrochloride and water without a stabilizer has less than about 10%, less than about 5%, or less than about 3%, of a naltrexone degradation product, as measured by HPLC, when stored at room temperature for 90 days. In some embodiments, the composition has less than about 10%, less than about 5%, or less than about 3%, of a naltrexone degradation product, as measured by HPLC, when stored at room temperature for 90 days, where the naltrexone degradation product is selected from the group consisting of 10-hydroxynaltrexone; 10-ketonaltrexone; 2,2 bisnaltrexone (pseudonaltrexone); oxides of 2,2 bisnaltrexone; dioxides of 2,2 bisnaltrexone; aldol adduct of naltrexone and 10-hydroxynaltrexone; aldol adduct of naltrexone and 10-ketonaltrexone; naltrexone-N-oxide; 10-hydroxynaltrexone-N-oxide; 10-ketonaltrexone-N-oxide; semiquinones of naltrexone; free radical peroxides of naltrexone; aldol adduct of naltrexone; aldol adducts of naltrexone coupled at the 7,6 position; aldol adducts of naltrexone coupled at the 6,5 position; ether-linked adduct of naltrexone; ether-linked adduct of naltrexone and 10-hydroxynaltrexone; ether-linked adduct of naltrexone and 10-ketonaltrexone; dehydrogenated naltrexone; hydroxy-naltrexone; keto-naltrexone; salts thereof and mixtures thereof.

In some embodiments, the intranasal composition comprising naltrexone hydrochloride and water without a stabilizer has greater than about than about 90%, or greater than about 95%, of the naltrexone hydrochloride in undegraded from, as measured by HPLC, when stored at room temperature for at least 90 days, and at least 180 days. Surprisingly, a composition consisting of naltrexone hydrochloride and water maintained greater than about 95% of naltrexone in an undegraded form, as measured by HPLC, over a period of at least 180 days when stored at room temperature.

Methods.

A method for treating a side effect or overdose of an opioid receptor agonist is provided comprising transmucosal administration of a composition to a patient in need thereof, wherein the composition comprises naltrexone, or a pharmaceutically acceptable salt thereof, and water.

The amount of naltrexone required for use in treatment will vary not only with the particular salt selected but also with the transmucosal route of administration, the nature of the condition being treated and the age and condition of the patient. The dosage will be at the discretion of the administering physician, emergency medical technician, or other clinician. In general, a suitable dose will be in the range of from about 0.001 mg/kg/day to about 10 mg/kg/day For example, a dosage may be from about 0.005 mg/kg to about 5 mg/kg of body weight per day, from about 0.02 mg/kg/day to about 1 mg/kg/day, and from about 0.01 mg/kg/day to about 0.1 mg/kg/day.

In some embodiments, the composition is administered in unit dosage form, e.g, containing from about 0.1 mg to about 10 mg, 0.5 mg to 5 mg, 1 mg-3 mg, or about 2 mg naltrexone hydrochloride per unit dosage form.

The desired dose may administered as a single dose or as divided doses administered at appropriate intervals, for example, as two, three, four or more sub-doses per day. Dosages above or below the range cited herein above are within the scope of the present invention and may be administered to the individual patient if desired and necessary.

A method for treating a side effect of an opioid receptor agonist is provided comprising intranasally or sublingually administering a composition to a patient in need thereof, wherein the patient is administered from about 0.1 mg to about 10 mg, 0.5 mg to 5 mg, 1 mg-3 mg, or about 2 mg naltrexone hydrochloride per dose of a composition consisting essentially of naltrexone hydrochloride and water.

In some specific embodiments, a method for treating or preventing an opioid receptor agonist side effect is provided comprising sublingually administering a composition consisting essentially of naltrexone hydrochloride and water. In some embodiments, the sublingual composition comprises 500 mcg/mL (0.5 mg/mL) naltrexone hydrochloride and the patient is sublingually administered from about 0.1 mL (50 mcg naltrexone hydrochloride) to about 0.2 mL (100 mcg naltrexone hydrochloride) from about 2 to about 6, or about 3 to 4 times per day.

In some specific embodiments, a method for treating or preventing an opioid receptor agonist side effect of constipation is provided comprising sublingually administering a composition consisting essentially of naltrexone hydrochloride and water, the sublingual composition comprising 500 mcg/mL (0.5 mg/mL) naltrexone hydrochloride and the patient is sublingually administered from about 0.1 mL (50 mcg naltrexone hydrochloride) to about 0.2 mL (100 mcg naltrexone hydrochloride) from about 2 to about 6, or about 3 to 4 times per day.

A method for treating an opioid receptor agonist overdose is provided comprising intranasally administering a composition to a patient in need thereof, wherein the patient is administered from about 0.1 mg to about 10 mg, 0.5 mg to 5 mg, 1 mg-3 mg, or about 2 mg naltrexone hydrochloride per dose of a composition consisting essentially of naltrexone hydrochloride and water.

As shown in the Examples, following intranasal administration of about 2 mg naltrexone hydrochloride to a patient, a blood sample obtained from a treated patient exhibits average blood levels of naltrexone appear to decline after about 30 minutes post-administration; however, average blood levels of naltrexone remain above about 2 ng/mL from at least about 15 minutes to about 60-120 minutes post-administration.

In some embodiments, the disclosure provides a method of treating a patient suspected of or known to be suffering from an opioid overdose comprising administering a first dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg of a centrally acting opioid receptor antagonist. In some embodiments, the dose is administered in a volume of about 0.1 mL, 0.3 mL, 0.5 mL, 0.7 mL, 1 mL, 1.3 mL, 1.5 mL, 2.0 mL, 2.5 mL, 3.0 mL, 3.5 mL, or 4.0 mL of a composition comprising an opioid receptor antagonist and water by intranasal spray administration. In some embodiments, the method further comprises administering a second dose of the composition by intranasal spray administration of the composition to the patient, if no response is detected after about 1, 2, 3, 4, 5, 6, 7, 8, 9, 10, 15, 20, 25, or 30 minutes following administration of the first dose; wherein the second dose is the same as, or greater than the first dose. In some embodiments, the opioid receptor antagonist is in the form of a pharmaceutically acceptable salt. In some embodiments, the composition consists of naltrexone hydrochloride and water.

In some embodiments, the disclosure provides a method of treating a patient suffering from an opioid receptor agonist overdose, a suspected opioid receptor agonist overdose, Crohn's disease, irritable bowel syndrome, fibromyalgia, neuropathic pain, chronic headache, postoperative dental pain, or an opioid side effect selected from constipation, drowsiness, nausea and vomiting, or pruritis; wherein the method comprises a step of administering a composition containing a pharmaceutically effective amount of an opioid receptor antagonist by intranasal administration.

In some embodiments, the disclosure provides a method of treating a patient in need thereof comprising administering a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg of naltrexone hydrochloride in water.

The compositions may be employed as follows. Nostrils of a patient known to be suffering or suspected of opioid overdose are inspected for mucous, blood or other problems that might inhibit absorption. A syringe is selected containing an appropriate dose of the intranasal composition comprising naltrexone hydrochloride (2 mg/mL for an adult or 0.1 mg/kg up to 2 mg single dose for a pediatric patient). Any air, if present, is expelled from syringe. A mucosal atomization device (MAD) is attached to the syringe via luer lock. The syringe plunger is briskly compressed to create a rapid intranasal mist spray of about 0.5 or 1 mL per nostril. In some cases, the entire dose is administered to a single nostril, or the dose may be split between nostrils. If no response is obtained in 3-5 minutes a second dose can optionally be administered. In some cases, a first dose is administered to one nostril. If no adequate response is detected, a second dose can be administered to the other nostril. Optimally, the patient will exhibit a gradual increase in consciousness with adequate respiratory efforts.

Kits.

In some embodiments, the compositions are provided in a kit form. In some embodiments, the kit comprises one or more single dose containers filled with a composition consisting essentially of naltrexone hydrochloride and water, and a sheet of instructions. In some embodiments, the single dose container is selected from a syringe or a vial. In some embodiments, each container holds enough composition such that a single delivered dose of naltrexone hydrochloride is selected from about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg of naltrexone hydrochloride in water. In some embodiments, the kit further comprises one or more mucosal atomization devices.

Mucosal Atomization Device.

In some embodiments, the kit comprises one or more single dose containers filled with the composition, a sheet of instructions, and one or more mucosal atomization devices (MADs). In some embodiments, one MAD is pre-fitted to each single dose container. In some embodiments, the kit comprises one or more MADs capable of being fitted to the single dose container(s) prior to use. In some embodiments, the MAD can be fitted to a syringe, or an MAD with syringe can be used in conjunction with a filled vial. Any mucosal atomization device (MAD) capable of being fitted to a syringe, e.g., fitted with a luer lock, can be employed. MADs are available commercially and include LMA/MAD Nasal™ intranasal mucosal atomization device (LMA North America, Inc, San Diego, Calif.), and Wolfe-Tory Mucosal Atomization Device MAD (Wolfe-Tory Medical, Salt Lake City, Utah).

### EXAMPLES

### Example 1

### Intranasal Composition

A Naltrexone hydrochloride 2 mg/mL nasal spray composition is prepared as described. Naltrexone hydrochloride USP anhydrous is dissolved in Water for injection with stirring. The amount of water for injection that is approximately 95% of the final volume is employed. For a final volume of 100 mL, 95 mL of water for injection is used. The solution is brought to the final volume with the addition of water for injection and mixed well. The solution is filtered through a 0.22 micron filter (PCCA #35-1156 or PCCA#35-1945) into sterile serum bottles or single use syringes with mucosal atomization device attached. Each mL contains 2 mg or 0.2% naltrexone hydrochloride. The dispensed solution is stored in refrigerator and protected from light.

### Example 2

### Stability of Naltrexone HCl in Aqueous Solution without Buffer or Preservative

USP Methodologies were used to evaluate the stability of Naltrexone hydrochloride compositions. The studies employed USP Methods: USP <621> HPLC Methodology; USP <85> Turbidometric or Photometric Technique; and USP <71> Membrane Filtration procedure, includes tests for Aerobic and Anaerobic bacteria, and Fungi (Molds and Yeasts), each of which is incorporated herein by reference.

Stability Test A: A 2.00 mg/mL solution of Naltrexone HCl in sterile water was prepared by dissolution of naltrexone HCl in water then q.s. No buffer or preservative was employed. The solution was stored at room temperature in 6×1 mL syringes and a 3 mL vial. Materials were maintained in the original container closure systems, under controlled temperature and humidity conditions per USP and exposed to ambient light (similar to normal exposure conditions) during storage and testing. The solution was analyzed for potency/purity at intervals over a 90 day period by USP <621> HPLC methodology. Results are shown in Table 1. The solution was evaluated for endotoxin at 90 days and found to be negative by USP <85> Turbidometric or Photometric technique. The solution was assayed for sterility (bacteria/fungi) at 14 and 90 days by USP<71> membrane Filtration Procedure, including tests for Aerobic and Anaerobic Bacteria, and Fungi (Molds and Yeasts), and found to be negative at 14 days and 5 days, respectively.

TABLE 1
Stability of Naltrexone HCl aqueous solution over 90 days.

| Test | Days | Specification | Result | Comment |
|---|---|---|---|---|
| Potency/Purity | 5 | 90.0-110.0% | 99.89% | Naltrexone HCl |
| | | | (2.00 mg/mL) | |
| Potency/Purity | 14 | 90.0-110.0% | 104.12% | Naltrexone HCl |
| | | | (2.08 mg/mL) | |
| Potency/Purity | 31 | 90.0-110.0% | 95.94% | Naltrexone HCl |
| | | | (1.92 mg/mL) | |
| Potency/Purity | 62 | 90.0-110.0% | 107.00% | Naltrexone HCl |
| | | | (2.14 mg/mL) | |
| Potency/Purity | 90 | 90.0-110.0% | 107.09% | Naltrexone HCl |
| | | | (2.14 mg/mL) | |
| Endotoxin | 90 | Report Results | <0.85 EU/mL | Meets |
| | | | | specification |
| Sterility | 6 | Negative at 14 | Negative at 14 | Meets |
| (Bacteria/ | | days | days | specification |
| Fungi) | | | | |
| Sterility | 90 | Negative at 14 | Negative at 5 | |
| (Bacteria/ | | days | days | |
| Fungi) | | | | |

Stability Test B:

A second solution of 2.00 mg/mL Naltrexone HCl in sterile water was prepared, packaged and stored as described for Stability Test A. Materials were maintained in the original container closure systems, under controlled temperature and humidity conditions per USP and were exposed to ambient light (similar to normal exposure conditions) during storage and testing. The solution was analyzed for potency/purity at intervals over a 180 day period by USP <621> HPLC methodology. Results are shown in Table 2. The naltrexone hydrochloride solution exhibited 90.0-110.0% Naltrexone and met specification for potency/purity over the entire test period of 180 days, when tested under USP<621>.

TABLE 2
Stability of Naltrexone HCl aqueous solution over 180 days.

| Test | Days | Specification | Result | Comment |
|---|---|---|---|---|
| Potency/ | 6 | 90.0-110.0% | 102.04% | Naltrexone HCl |
| Purity | | | (2.04 mg/mL) | Meets specification |
| Potency/ | 15 | 90.0-110.0% | 102.11% | Naltrexone HCl |
| Purity | | | (2.04 mg/mL) | Meets specification |
| Potency/ | 33 | 90.0-110.0% | 101.05% | Naltrexone HCl |
| Purity | | | (2.02 mg/mL) | Meets specification |
| Potency/ | 61 | 90.0-110.0% | 98.49% | Naltrexone HCl |
| Purity | | | (1.97 mg/mL) | Meets specification |
| Potency/ | 90 | 90.0-110.0% | 99.24% | Naltrexone HCl |
| Purity | | | (1.98 mg/mL) | Meets specification |
| Potency/ | 121 | 90.0-110.0% | 98.70% | Naltrexone HCl |
| Purity | | | (1.97 mg/mL) | Meets specification |
| Potency/ | 152 | 90.0-110.0% | 98.56% | Naltrexone HCl |
| Purity | | | (1.97 mg/mL) | Meets specification |
| Potency/ | 180 | 90.0-110.0% | 100.44% | Naltrexone HCl |
| Purity | | | (2.01 mg/mL) | Meets specification |
| Endotoxin | 6 | ≦1.84 EU/mL | <0.50 EU/mL | Meets specification |
| Endotoxin | 180 | Report Results | <1.14 EU/mL | |
| Sterility | 6 | Negative at 14 | Negative at 14 | Meets specification |
| (Bacteria/ | | days | days | |
| Fungi) | | | | |

The solution was analyzed for endotoxin at 6 and 180 days by USP <85> Turbidometric or Photometric technique. The solution met specifications for ≦1.84 EU/mL over the test period of 180 days when tested under USP <85>.

The solution was analyzed for sterility (bacteria/fungi) at 6 and 14 days by USP<71> membrane Filtration Procedure, including tests for Aerobic and Anaerobic Bacteria, and Fungi (Molds and Yeasts), and found to be negative at both 6 days and 14 days. The solution met specifications for sterility when tested for bacteria/fungi under USP<71>.

Surprisingly, the naltrexone HCl solution was stable without an antioxidant, surfactant, adhesive, pH adjusting agent, stabilizer, osmolarity adjusting agent, tonicity agent, preservative, thickening agent, buffering agent, chelating agent, or penetration enhancer for a period of 180 days when stored at room temperature.

### Example 3

### Blood Levels of Free Naltrexone and Metabolite Free 6-Beta-Naltrexol Following Intranasal Administration

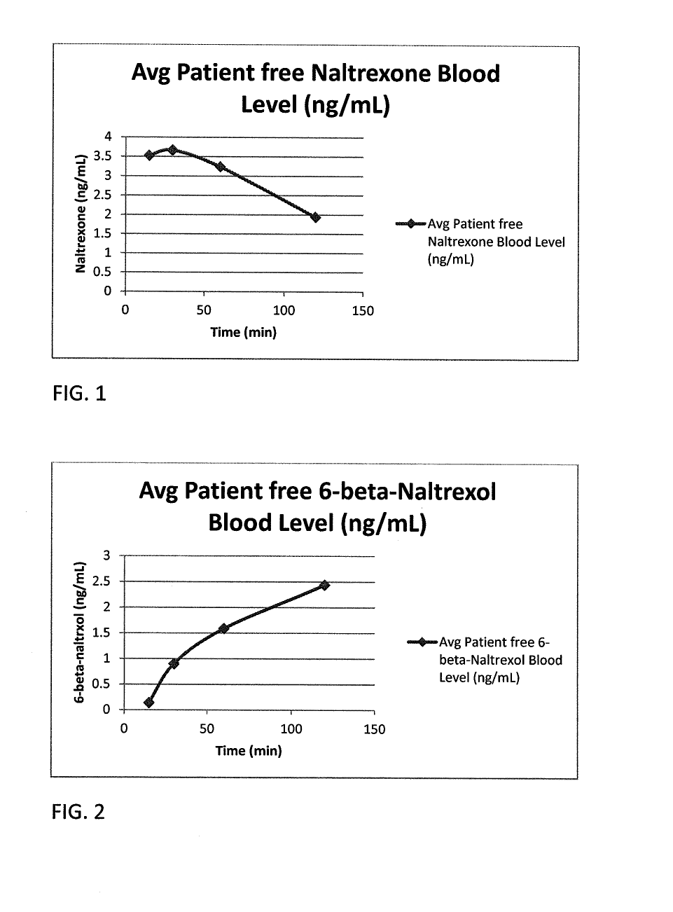

The composition of Example 1 (one mL) was administered as an intranasal spray to eight adult volunteers; three males and five females. Blood samples were obtained and processed from each volunteer at 15, 30, 60 and 120 minutes following intranasal administration. HPLC was used to determined blood levels of free naltrexone (ng/mL) and metabolite free 6-beta-naltrexol. Data for free Naltrexone in patient blood samples is shown in Table 3. A graph of average blood levels of free naltrexone over time following intranasal administration are exhibited in FIG. 1. FIG. 1 shows a graph of average blood levels from Table 3 of free Naltrexone (ng/mL) of 8 subjects at various time points (15, 30, 60 and 120 minutes) following intranasal administration of 1 mL of the composition according to Example 1, consisting essentially of naltrexone hydrochloride and water. Average blood levels of naltrexone peak sometime between 15 and 60 minutes post-intranasal administration. Average blood levels of naltrexone remain above about 2 ng/mL from at least between about 15 minutes to about 60-120 minutes post-intranasal administration.

TABLE 3
Patient Blood Level of Naltrexone following Intranasal Administration.
Naltrexone-free (ng/mL) blood levels-Intranasal administration

| Patient | 15 min | 30 min | 60 min | 120 min |
|---|---|---|---|---|
| 1 | 1.6 | 2.3 | 1.8 | 1.3 |
| 2 | 4.6 | 5.5 | 4.5 | 2.1 |
| 3 | 2.1 | 2.8 | 2.6 | 1.4 |
| 4 | 4 | 3.4 | 2.9 | 1.8 |
| 5 | 5.2 | 5.1 | 4.6 | 2.8 |
| 6 | 1.4 | 1.7 | 1.8 | 1.1 |
| 7 | 4.7 | 4.7 | 4.5 | 2.9 |
| 8 | 4.6 | 3.8 | 3.2 | 2.1 |
| Avg. | 3.525 | 3.663 | 3.238 | 1.938 |
| Std. Dev. | 1.457 | 1.278 | 1.101 | 0.626 |

Table 4 shows blood levels of 6-beta-naltrexol following intranasal administration of naltrexone hydrochloride composition of Example 1. Table 4. Patient Blood Level of 6-beta-Naltrexol following Intranasal Administration.

TABLE 4
Blood Level 6-beta-Naltrexol-free (ng/mL)

| Patient | 15 min | 30 min | 60 min | 120 min |
|---|---|---|---|---|
| 1 | 0 | 1 | 1.2 | 2 |
| 2 | 0 | 1.5 | 1.6 | 1.8 |
| 3 | 0 | 0 | 0 | 1 |
| 4 | 0 | 1.9 | 2.9 | 3.5 |
| 5 | 1.1 | 1.8 | 4 | 4.3 |
| 6 | 0 | 0 | 1.1 | 2.1 |
| 7 | 0 | 1 | 1.9 | 2.1 |
| 8 | 0 | 0 | 0 | 2.7 |
| Avg. | 0.138 | 0.900 | 1.588 | 2.438 |
| Std. Dev. | 0.364 | 0.760 | 1.278 | 0.972 |

FIG. 2 shows a graph of average blood levels from Table 4 of free 6-beta-naltrexol (ng/mL), a naltrexone metabolite, of 8 subjects at various time points (15, 30, 60 and 120 minutes) following intranasal administration of 1 mL of the composition according to Example 1, consisting essentially of naltrexone hydrochloride and water. Average blood levels of 6-beta-naltrexol increase over time to an average of about 2.4 ng/mL at 120 minutes post-intranasal administration.

We claim:

1. A pharmaceutical composition consisting essentially of naltrexone hydrochloride and water, the composition capable of maintaining at least about 90% of said naltrexone hydrochloride in undegraded form after storage for 180 days at room temperature.

2. The composition of claim 1 wherein the composition is capable of maintaining at least about 95% of said naltrexone in undegraded form after storage for 180 days at room temperature.

3. The composition of claim 1 wherein the naltrexone hydrochloride is present in the composition in an amount between about 0.001 mg/mL and 20 mg/mL.

4. The composition of claim 1 wherein the naltrexone hydrochloride is present in the composition in an amount between 0.5 mg/mL and 10 mg/mL.

5. The composition of claim 1 wherein the naltrexone hydrochloride is present in the composition in an amount between 1 mg/mL and 3 mg/mL.

6. The composition of claim 1 wherein the naltrexone hydrochloride is present in the composition in about 2 mg/mL.

7. The composition of claim 1 wherein the composition consists of naltrexone hydrochloride and water.

8. The composition of claim 1 wherein the composition does not contain a preservative.

9. The composition of claim 1 wherein the composition does not contain a buffering agent.

10. The composition of claim 1 wherein the composition does not contain a tonicity agent.

11. The composition of claim 1 wherein the composition does not contain a pH adjusting agent.

12. The composition of claim 1 wherein the composition consists of naltrexone hydrochloride dissolved in water at a concentration in the range of from about 0.001 mg/mL to about 20 mg/mL, about 0.05 mg/mL to about 10 mg/mL, about 0.5 mg/mL to about 5 mg/mL, about 1 mg/mL to about 3 mg/mL, or about 2 mg/mL in sterile water or water for injection.

13. A method for treating a patient in need thereof, the method comprising
administering transmucosally to the patient a first dose of a pharmaceutical composition consisting essentially of a pharmaceutically effective dose of naltrexone hydrochloride and water; wherein the composition is capable of maintaining at least about 90% of said naltrexone hydrochloride in undegraded form after storage for 180 days at room temperature.

14. The method of claim 13, wherein the administering transmucosally is elected from administering intranasally, buccally, sublingually, vaginally, ocularly or rectally.

15. The method of claim 13, wherein the administering intranasally comprises administering the composition as an intranasal spray or an intranasal drop.

16. The method of claim 13, further comprising
administering a second dose of the composition intranasally to the patient, if no response is detected following administration of the first dose.

17. The method of claim 16 wherein the second dose is administered if no response is detected following administration of the first dose within a range of from about 1 minutes to about 10 minutes following administration of the first dose.

18. The method of claim 16, wherein the second dose of the composition is the same or greater than amount of naltrexone hydrochloride as, the first dose.

19. The method of claim 13, wherein the patient in need thereof is suffering from an opioid receptor agonist overdose, a suspected opioid receptor agonist overdose, Crohn's disease, irritable bowel syndrome, fibromyalgia, neuropathic pain, chronic headache, postoperative dental pain, or an opioid side effect selected from constipation, drowsiness, urinary retention, nausea, vomiting, headache, or pruritis.

20. The method of claim 13, wherein the patient in need thereof is suffering from, or suspected of suffering from, an opioid agonist overdose.

21. A kit comprising:
one or more sealed containers, each container filled with a composition consisting essentially of naltrexone hydrochloride and water, the composition capable of maintaining at least about 90% of said naltrexone hydrochloride in undegraded form after storage for 180 days at room temperature, and
a sheet of instructions.

22. The kit of claim 21 wherein the composition does not contain a buffering agent.

23. The kit of claim 21, wherein the kit further comprises one or more mucosal atomization devices (MAD), wherein the MADs are fitted to, or capable of being fitted to, the one or more containers for intranasal spray administration of the composition to a patient in need thereof.
